(19) United States
(12) Patent Application Publication
(10) Pub. No.: US 2007/0254327 A1
(43) Pub. Date: Nov. 1, 2007

(54) Method for Performing the Hot Start of Enzymatic Reactions

(76) Inventors: Konstantin Ignatov, Moscow (RU); Vladimir Kramarov, Moscow (RU)

Correspondence Address: NIXON & VANDERHYE, PC, 901 NORTH GLEBE ROAD, 11TH FLOOR, ARLINGTON, VA, 22203, US

(21) Appl. No.: 11/632,700

(22) PCT Filed: Jul. 14, 2005
(86) PCT No.: PCT/GB05/02774

§ 371 Date: Mar. 14, 2007

Related U.S. Application Data
(60) Provisional application No. 60/589,591, filed on Jul. 21, 2004.

(30) Foreign Application Priority Data
Jul. 21, 2004 (GB) 0416293.9

(51) Int. Cl. C12Q 1/68

(52) U.S. Cl. 435/25; 435/183

(57) ABSTRACT

The present invention provides processes for controlling the start of an enzymatic reaction, which is catalysed by a metal ion dependent enzyme. The required metal ion is generated by a redox reaction initiated by heating a metal compound having a metal atom or metal ion with a redox agent. Also provided are kits for controlling the start of an enzymatic reaction. The processes and kits of the invention are useful for improving the specificity and performance of PCR.

### FIELD OF THE INVENTION

The present invention provides processes and kits for controlling the start of an enzymatic reaction. A metal-ion dependent enzyme catalyses the enzymic reaction, with the required metal ion generated by a redox reaction. The processes of the present invention are useful for improving the specificity and performance of PCR.

### BACKGROUND

The present invention provides a method for performing an enzymatic reaction, which is catalyzed by a metal-ion dependent enzyme (e.g., a restriction endonuclease, a DNA ligase, a reverse transcriptase or a DNA dependent DNA polymerase).

In biomolecular processes it is often important to control the activity of an enzyme. This is particularly the case with DNA polymerase enzymes used for the polymerase chain reaction (PCR). PCR reactions often involve the use of a divalent metal ion-dependent heat-resistant DNA polymerase enzyme (such as Tag DNA polymerase) in a multi-cycle process employing several alternating heating and cooling steps to amplify DNA (U.S. Pat. Nos. 4,683,202 and 4,683,195). First, a reaction mixture is heated to a temperature sufficient to denature the double stranded target DNA into its two single strands. The temperature of the reaction mixture is then decreased to allow specific oligonucleotide primers to anneal to their respective complementary single stranded target DNAs. Following the annealing step, the temperature is raised to the temperature optimum of the DNA polymerase being used, which allows incorporation of complementary nucleotides at the 3′ ends of the annealed oligonucleotide primers thereby recreating double stranded target DNA. Using a heat-stable DNA polymerase, the cycle of denaturing, annealing and extension may be repeated as many times as necessary to generate a desired product, without the addition of polymerase after each heat denaturation. Twenty or thirty replication cycles can yield up to a million-fold amplification of the target DNA sequence (“Current Protocols in Molecular Biology,” F. M. Ausubel et al. (Eds.), John Wiley and Sons, Inc., 1998).

Although PCR technology has had a profound impact on biomedical research and genetic identity analysis, amplification of non-target oligonucleotides and mispriming on non-target background DNA, RNA, and/or the primers themselves, still presents a significant problem. This is especially true in diagnostic applications where PCR is carried out in a milieu of complex genetic backgrounds where the target DNA may be proportionately present at a very low level (Chou et al., Nucleic Acid Res., 20:1717-1723 (1992).

A chief problem is that even though the optimal temperature for Taq DNA polymerase activity is typically in the range of 62°-72° C., significant activity can also occur between 20°-37° C. (W. M. Barnes, et al, U.S. Pat. No. 6,403,341). As a result, during standard PCR preparation at ambient temperatures, primers may prime extensions at non-specific sequences because only a few base pairs at the 3′-end of a primer which are complementary to a DNA sequence can result in a stable priming complex. As a result, competitive or inhibitory products can be produced at the expense of the desired product. Thus, for example, structures consisting only of primers, sometimes called “primer dimers” can be formed by Taq DNA polymerase activity on primers inappropriately paired with each other.

The probability of undesirable primer-primer interactions also increases with the number of primer pairs in a reaction, particularly in the case of multiplex PCR. Mispriming of template DNA can also result in the production of inhibitory products or “wrong bands” of various lengths. During PCR cycling, non-specific amplification of undesired products can compete with amplification of the desired target DNA for necessary factors and extension constituents, such as dNTPs, which can lead to misinterpretation of the assay. Given the sensitivity of Taq DNA polymerase and its propensity to progressively amplify relatively large amounts of DNA from any primed event, it is imperative to control Taq DNA polymerase activity to prevent production of irrelevant, contaminating DNA amplification products, particularly when setting up PCR reactions.

Undesirable PCR side reactions typically occur during PCR preparation at ambient temperatures. One approach for minimizing these side reactions involves excluding at least one essential reagent (dNTPs, Mg²⁺, DNA polymerase or primers) from the reaction until all the reaction components are brought up to a high (e.g., DNA denaturation) temperature; the idea is to prevent binding of primers to one another or to undesired target sequences (Erlich, et al, Science 252, 1643-1651, 1991; D'Aquila, et al, Nucleic Acids Res. 19, 3749, 1991). This is an example of a “physical” PCR hot-start approach where an essential component is physically withheld until a desired reaction temperature is reached.

Other hot-start approaches have been described that physically segregate the reaction components from each other to guarantee that DNA polymerase activity is suppressed during the period preceding PCR initiation. In this way, a physical segregation of a hot start can be achieved by using a wax barrier, such as the method disclosed in U.S. Pat. Nos. 5,599,660 and 5,411,876. See also Hebert et al., Mol. Cell Probes, 7:249-252 (1993); Horton et al., Biotechniques, 16:42-43 (1994).

Other hot-start approaches have been described that employ the “chemical/biochemical hot-start” methods that utilize modified DNA polymerases reversibly activatable upon heating (e.g., AMPLITAQ GOLD™ DNA POLYMERASE, PE Applied Biosystems) or monoclonal, inactivating antibodies against Taq DNA polymerase that are bound to the polymerase at ambient temperatures (Scalice et al., J. Immun. Methods, 172: 147-163, 1994; Sharkey et al., Bio/Technology, 12:506-509, 1994; Kellogg et al., Biotechniques, 16: 1134-1137, 1994).

The aforementioned different PCR hot-start approaches have multiple shortcomings. Physical hot-start methods are plagued by contamination problems, plugging up of pipet tips with wax or grease and increased heating times. Chemical/biochemical hot-start methods can damage the template DNA and can require prohibitively excessive amounts of expensive anti-Amplitaq™ antibodies.

Accordingly, there is a need in the art for new PCR hot-start methods minimizing or eliminating the many problems or shortcomings associated with the prior art procedures. More generally, there is a need for new approaches for controlling metal-ion dependent enzymes where controlled activity is desired.

### SUMMARY OF INVENTION

The present invention provides processes and reaction kits for initiating an enzymatic reaction catalysed by a metal ion-dependent enzyme.

A process of the invention may comprise the steps of:
a) providing a reaction mixture comprising
   i) a metal compound having a metal atom or metal ion in a first oxidation state;
   ii) a redox agent; and
   iii) a metal ion-dependent enzyme;
b) heating the mixture of step (a) to react the metal compound with the redox agent in a redox reaction, thereby converting the metal atom or metal ion to a second oxidation state;

wherein, the metal ion-dependent enzyme is activated by the metal atom or metal ion in the second oxidation state.

In one embodiment of the present invention, the first oxidation state of the metal atom or metal ion in the metal compound may be an oxidized state. The second oxidation state of the metal atom or metal ion may be a reduced state. The redox agent is a reducing agent.

In an alternative embodiment, the first oxidation state of the metal atom or metal ion in the metal compound may be a reduced state. The second oxidation state of the metal atom or metal ion may be an oxidized state. The redox agent is an oxidizing agent.

The redox reaction that generates the metal atom or metal ion in a second oxidation state can occur in a controlled manner, depending on physical conditions. These conditions include temperature and incubation time. Preferably the reaction mixture is heated to a temperature greater than 50° C. In effect, the redox reaction can provide a controlled generation of an essential metal ion and as a result, controlled initiation of an enzymatic process catalysed by a metal ion-dependent enzyme.

The metal atom or metal ion in the second oxidation state may include a monovalent, divalent or polyvalent metal ion from one of cobalt, manganese, cadmium, copper, iron, molybdenum, nickel or chromium. Preferably the metal atom or metal ion in the second oxidation state is a divalent ion. More preferably the metal ion in the second oxidation state is Co²⁺.

The reaction generating the metal ion in the second oxidation state can be a redox reaction, such as a reduction of cobalt(III) to cobalt(II), or a similar reaction such as the reduction of iron(III) to iron(II), chromium(VI) or chromium(III) to chromium(II), manganese(VII) or manganese(IV) to manganese(II).

In an embodiment of the present invention, the metal ion dependent enzyme may be selected from: a polymerase, a ligase, an endonuclease, a kinase, a protease or a combination thereof. Preferably the enzyme is a thermostable enzyme such as DNA ligase or DNA polymerase. Where the enzyme is DNA polymerase, the enzyme is preferably Taq polymerase or a variant thereof.

The enzymatic reaction according to the present invention may comprise a PCR process.

A further embodiment of the present invention relates to kits for use in the processes described above. A kit according to the present invention may comprise a number of components required to generate the metal atom or metal ion in a second oxidation state necessary for activating the metal ion-dependent enzyme and initiating the enzymatic process of the invention. The kits may be suitable for use in PCR reactions. The reaction components may be stored separately to avoid unwanted initiation of a redox reaction.

Other features, aspects and advantages of the invention will be, or will become, apparent to one with skill in the art upon examination of the following figures and detailed description. It is intended that all such additional systems, features, aspects and advantages included within this description, are within the scope of the invention, and are protected by the following claims.

### BRIEF DESCRIPTION OF THE FIGURES

These and other features, aspects, and advantages of the present invention will become better understood with regard to the following description, claims and accompanying drawings where:

FIG. 1 depicts an electrophoretic analysis of the PCR products obtained in Example 2 using conventional PCR with ordinary PCR-buffer containing Mg²⁺ (lane 1) or Co²⁺ (lane 2), or using gPCR with controlled generation of Co²⁺ (lane 3). Lane 4—DNA marker.

FIG. 2 depicts an electrophoretic analysis of DNA fragments obtained following restriction endonuclease digestion of pBR322 using TaqI as described in Example 3. The enzymatic reaction was performed with (lane 2) and without (lane 1) heat initiation of Co²⁺ generation. Lane 3—positive control of endonuclease digestion in presence of Co²⁺ (conventional endonuclease digestion).

### DETAILED DESCRIPTION

In order to provide a clear and consistent understanding of the specification and claims, the following definitions are provided.

“Metal atom or metal ion” is used herein to designate a metal atom or metal ion, which as a result of a redox reaction, undergoes a change in its oxidation state, thereby generating a metal ion necessary for activating a metal ion-dependent enzyme. The metal atom or metal ion ma y be selected from atoms and ions of cobalt, manganese, cadmium, copper, iron, molybdenum, nickel or chromium. The metal ion may comprise a monovalent, divalent or polyvalent metal ion.

“Thermostable”, “thermally stable” and “heat-stable” are used interchangeably herein to describe enzymes, which can withstand temperatures up to at least 95° C. for several minutes without becoming irreversibly denatured. Typically, such enzymes have an optimum temperature above 45° C., preferably between 50° to 75° C.

“Hot start” refers to the method of initiating an enzymic reaction by heating components of the reaction. The reaction components may be heated to a specific temperature or to a range of temperatures.

The term “redox” refers to reduction-oxidation, a term that is well known in the art, in which reduction is gain of electrons and oxidation is loss of electrons.

A “metal compound” describes a metal atom or ion in combination with another element or compound, for example, in combination with chlorine or sulphate to give a metal chloride or metal sulphate. Formation of the metal compound involves a chemical reaction. Also encompassed within this definition are metal complexes or coordination compounds in which other atoms or ligands are bound to a central metal ion. The ligands may be negatively charged or strongly polar groups.

A metal atom in a first oxidation state describes a metal atom in a compound, in which the atom has an overall charge of zero i.e. the number of electrons equals the number of protons. A metal atom in a second oxidation state describes a metal atom which posses a different number of electrons to the number it possessed in the first oxidation state i.e. the metal atom in a second oxidation state is a metal ion.

A metal ion in a first oxidation state describes a metal ion in a compound, in which the ion is in a reduced or oxidized state. A metal ion in a second oxidation state describes a metal ion which posses a different number of electrons to the number it possessed in the first oxidation state.

A redox reaction accounts for the transfer of electrons to or from the metal atom or metal ion in its first oxidation state to its second oxidation state. When the first oxidation state is a reduced state, the second oxidation state will be an oxidized state. When the first oxidation state is an oxidized state, the second oxidation state will be a reduced state.

The present invention provides processes for performing a metal ion-dependent enzymatic reaction in which required metal ions arise as a result of a non-enzymatic redox reaction. Generation of the metal ion by the redox reaction is determined by physical conditions of the reaction, such as temperature and incubation time. Thus, the redox reaction can provide a controlled generation of an essential metal ion. By controlling the generation of the metal ion, the present invention provides a means for controlling enzymatic processes, including, but not limited to, the start of an enzymatic process.

The redox reaction may provide a controlled generation of a metal ion, such as Co²⁺. Preferably the redox reaction is the reduction of cobalt(III) to cobalt(II). For controlled generation of other metal ions, such as Fe²⁺, Cr²⁺ or Mn²⁺, similar reactions can be used (e.g., reactions of reduction of iron(III) to iron(II), chromium(VI) or chromium(III) to chromium(II), manganese(VII) or manganese(IV) to manganese(II), and others). As a reducing agent in these reactions ascorbic acid may be used, or potassium or sodium iodide, potassium or sodium thiosulfate or other reactants.

Preferred chemical reactions for generation of Co²⁺ as a metal ion for use with cobalt-dependent enzymes, include, but are not limited to reactions of reduction of cobalt(III) to cobalt(II) (e.g., [Co(NH₃)₆]³⁺+e⁻→Co²⁺+6NH₃).

Preferred chemical reactions for generation of Mn²⁺ as a metal ion for use with manganese-dependent enzymes, include, but are not limited to reactions of reduction of manganese(VII) or manganese(IV) to manganese(II) (e.g., MnO₄⁻+4H₂O+5e⁻→Mn²⁺+8OH⁻).

Preferred chemical reactions for generation of Cr²⁺ as a metal ion for use with chrome-dependent enzymes, include, but are not limited to reactions of reduction of chromium(VI) or chromium(III) to chromium(II) (e.g., CrO₄²⁻4H₂O+4e⁻→Cr²⁺+8OH⁻, or Cr³⁺+e⁻→Cr²⁺).

Preferred chemical reactions for generation of Cr³⁺ as a metal ion for use with chrome-dependent enzymes, include, but are not limited to reactions of reduction of chromium(VI) to chromium(III) and oxidation of chromium(II) to chromium(III) (e.g., CrO₄²⁻4H₂O+3e⁻→Cr³⁺+8OH⁻, and Cr²⁺−e⁻→Cr³⁺).

Preferred chemical reactions for generation of Fe²⁺ as a metal ion for use with iron-dependent enzymes, include, but are not limited to reactions of reduction of iron(III) to iron(II) (e.g., Fe³⁺+e⁻Fe²⁺).

Preferred chemical reactions for generation of Fe³⁺ as a metal ion for use with iron-dependent enzymes, include, but are not limited to reactions of oxidation of iron(II) to iron(III) (e.g., Fe²⁺−e⁻→Fe³⁺).

Preferred chemical reactions for generation of Cu²⁺ as a metal ion for use with copper-dependent enzymes, include, but are not limited to reactions of oxidation of copper(I) to copper(II) (e.g., Cu⁺−e⁻→Cu²⁺)

Preferred chemical reactions for generation of Cu⁺ as a metal ion for use with copper-dependent enzymes, include, but are not limited to reactions of reduction of copper(II) to copper(I) (e.g., Cu²⁺+e⁻→Cu⁺).

Preferred chemical reactions for generation of Ni²⁺ as a metal ion for use with nickel-dependent enzymes, include, but are not limited to reactions of reduction of nickel(III) to nickel(II) (e.g., Ni³⁺+e⁻→Ni²⁺, or Ni₂O₃+3H₂O+2e⁻→2Ni²⁺+6OH⁻).

Preferred metal compounds of cobalt(III) for use in redox reaction of Co²⁺ generation include, but are not limited to cobalt(III) complex compounds such as [Co(NH₃)₆]Cl₃, Na₃[Co(CN)₆] and others.

Preferred metal compounds of manganese(VII) and manganese(IV) for use in redox reaction of Mn²⁺ generation include, but are not limited to compounds such as KMnO₄, NaMnO₄, MnO₂, MnO(OH)₂, and others.

Preferred metal compounds of chromium(VI) and chromium(III) for use in redox reaction of Cr²⁺ generation include, but are not limited to compounds such as K₂CrO₄, (NH₄)₂CrO₄, Cr₂(SO₄)₃, CrCl₃, Cr(OH)₃, Cr(NO₃)₃ and others.

Preferred metal compounds of chromium(VI) and chromium(II) for use in redox reaction of Cr³⁺ generation include, but are not limited to compounds such as K₂CrO₄, (NH₄)₂CrO₄, CrCl₂, and others.

Preferred metal compounds of iron(III) for use in redox reaction of Fe²⁺ generation include, but are not limited to compounds such as NH₄Fe(SO₄)₂, FeCl₃, Fe(NO₃)₃, Fe₂(SO₄)₃ and others.

Preferred metal compounds of iron(II) for use in redox reaction of Fe³⁺ generation include, but are not limited to compounds such as (NH₄)₂Fe(SO₄)₂, FeCl₂, FeSO₄ and others.

Preferred metal compounds of copper(I) for use in redox reaction of Cu²⁺ generation include, but are not limited to compounds such as CuCl, CuI, CuSCN and others.

Preferred metal compounds of copper(II) for use in redox reaction of Cu⁺ generation include, but are not limited to compounds such as CuCl₂, CuBr₂, CuSO₄ and others.

Preferred metal compounds of nickel(III) for use in redox reaction of Ni²⁺ generation include, but are not limited to compounds such as CuCl₂, CuBr₂, CuSO₄ and others.

The above mentioned redox reactions, which provide for generation of an essential metal-ion and, as a result, for the start of a metal-ion dependent enzymatic process, can be initiated by heating a reaction mixture to a temperature over 50° C. Thus, the metal-ion dependent enzymatic process can be started in a controlled manner after heating the reaction mixture, thereby providing the hot-start of the enzymatic process.

The method of the invention may be applied to initiate or hot start metal-ion dependent enzymatic reactions which are catalyzed by DNA- and RNA-dependent DNA-polymerases, restriction endonucleases, DNA- and RNA-ligases, kinases, proteinases, and other metal-ion dependent enzymes. Particularly, the present invention can be used to initiate a PCR process.

The process of the present invention can increase the specificity of PCR reactions by preventing activation of a thermostable DNA polymerase (e.g. Taq DNA polymerase) at lower temperatures, while promoting temperature-dependent generation of divalent metal ions (e.g., generation of Co²⁺ or Mn²⁺ at 60-98° C.) and selection of specifically bound primers for DNA polymerase-catalyzed extension.

The PCR processes employ heat-stable DNA polymerase enzymes. These enzymes (e.g., Taq, Tth or Pfu DNA polymerase) are divalent metal ion-dependent enzymes. These polymerases require the presence of Mg²⁺, or Co²⁺, or Mn²⁺ as a metal ion cofactor for activation. In order to perform a hot-start PCR by the method of the present invention, a reaction that generates Co²⁺ ions by reduction of cobalt(III) to cobalt(II) can be used.

Preferred reducing chemical agents for reduction of cobalt(III) to cobalt(II) in redox reaction of Co²⁺ generation include, but are not limited to ascorbic acid, salts of ascorbic acid, hydroiodic acid, salts of hydroiodic acid such as potassium, sodium or ammonium iodide, potassium thiosulphate and sodium thiosulphate.

In order to perform a hot-start PCR, the redox reaction between hexamminecobalt(III) chloride and ascorbic acid can be used. Under PCR conditions, this redox reaction generates Co²⁺ ions only at temperatures over 50° C. Thus, the enzymatic process (PCR) is initiated by the redox reaction only after heating the reaction mixture to a temperature above 50° C. As a result, the specificity of PCR is enhanced.

In a similar, the reduction-oxidation reaction between potassium permanganate (KMnO₄) and ascorbic acid (C₆H₈O₆) may be used, in order to perform PCR process. Under PCR conditions, this reduction-oxidation reaction generates Mn²⁺ ions.

Metal ion-dependent enzymes that may be controlled in accordance with the present invention include a variety of enzyme members or species defined by the several generic enzyme classes, including DNA polymerases, RNA polymerases, reverse transcriptases, DNA ligases, endonucleases, restriction endonucleases, kinases, and proteases. Metal-ion dependent enzymes may originate from a wide variety of animal, bacterial or viral sources, and may be synthesized from native genetic structures or from variants genetically modified by e.g., mutagenesis or genetically modified to express fusion proteins, carrying multiple, distinct functional domains.

Additional examples of metal-ion dependent enzymes include DNA polymerases, such as Klenow fragment and DNA PolI; reverse transcriptases (RT), such as AMV RT and MMLV RT; most restriction endonucleases; ribonucleases, such as RNase H; and topoisomerases, such as Topoisomerase I.

Many enzymes can alternatively use a few different metal ions. For example, RNA polymerases, such as RNA polymerase I or T7-, SP6-, and T4 RNA polymerases can use Mg²⁺ or Mn²⁺. DNase I can utilize a variety of different metal ions, including Mg²⁺, Mn²⁺, Ca²⁺, Co²⁺ or Zn²⁺.

Enzymes for use in the present invention may be preferably selected or engineered on the basis of retaining enzymatic stability under a range of reaction conditions required by generation of ionic enzymatic reactants, including high temperatures and/or various pH conditions (high/low, etc.). Particularly preferred enzymes include thermostable and/or pH resistant enzymes.

Thermostable enzymes may be isolated from thermophilic bacterial sources (e.g., thermophilic genus Thermus) or they may be isolated and prepared by means of recombination. Representative species of the Thermus genus include T. aquaticus, T. thermophilus, T. rubber, T. filiformis, T. brockianus and T. scotoductus. The thermostable enzymes for use in the present invention may be derived from a broad range of enzyme types.

Examples of thermostable enzymes for use in the present invention, include, but are not limited to: thermostable DNA polymerases disclosed in e.g., U.S. Pat. Nos. 4,889,818, 5,079,352, 5,192,674, 5,374,553, 5,413,926, 5,436,149, 5,455,170, 5,545,552, 5,466,591, 5,500,363, 5,614,402, 5,616,494, 5,736,373, 5,744,312, 6,008,025, 6,027,918, 6,033,859, 6,130,045, 6,214,557; thermostable reverse transcriptases disclosed in e.g., U.S. Pat. No. 5,998,195 and U.S. 2002/0090618; thermostable phosphatases disclosed in e.g., U.S. Pat. Nos. 5,633,138, 5,665,551, 5,939,257; thermostable ligases disclosed in e.g., U.S. Pat. Nos. 5,494,810, 5,506,137, 6,054,564 and 6,576,453; thermostable proteases disclosed in e.g., U.S. Pat. Nos. 5,215,907, 5,346,820, 5,346,821, 5,643,777, 5,705,379, 6,143,517, 6,294,367, 6,358,726, 6,465,236; thermostable topoisomerases disclosed in e.g., U.S. Pat. Nos. 5,427,928 and 5,656,463; thermostable ribonucleases disclosed in e.g., U.S. Pat. Nos. 5,459,055 and 5,500,370; thermostable beta-galactosidases disclosed in e.g., U.S. Pat. Nos. 5,432,078 and 5,744,345; thermostable restriction endonucleases, including e.g., AccIII, AcsI/ApoI, AcyI, BcoI, BsaBI/BsiBI, BsaMI, BsaJI, BsaOI, BsaWI, BscBI, BscCI, BscFI, BseAI, BsiC1, BsiE1, BSi HKAJ, BsiLI, BsiMI, BsiQI, BsiWI, BsiXI, BsiZI, BsiI, BsmI, BsmAI, BsmBI, Bss, T11, Bsr1, BsrD1, Bsi711, BsiB1, BsiN1, BsiU1, BsiY1, BsiZ1, Dsa 1, Mae 11, Mae 111, Mwo 1, Ssp B1, TaqI, TaqII, Taq52 I, TfiI, Tru91, TspE1, TspRI, Tsp45 I, Tsp4C I, Tsp509 I, Tth111 II; Flap endonuclease disclosed in U.S. Pat. No. 6,251,649; and FLPe, a mutant, thermostable recombinase of Flp (Bucholz et al., Nature Biotechnology, Vol. 16, pp. 657-662, 1998).

Preferred metal ion-dependent enzymes include, but are not limited to thermally stable enzymes. Thermostable metal ion-dependent enzymes may include thermostable DNA polymerases, RNA polymerases, reverse transcriptases, DNA ligases, endonucleases, restriction endonucleases, kinases, and proteases, including, but not limited to the aforementioned enzymes above. Thermally stable enzymes may be isolated from thermophilic bacterial sources or they may be isolated and prepared by recombinant means.

Preferred DNA polymerases for use in PCR applications include thermally stable DNA polymerases and/or combinations thereof. Thermally stable DNA polymerases may include, but are not limited to, Thermus aquaticus DNA polymerase and variations thereof such as N-terminal deletions of Taq polymerase, including the Stoffel fragment of DNA polymerase, Klentaq-235, and Klentaq-278; Thermus thermophilus DNA polymerase; Bacillus caldotenax DNA polymerase; Thermus flavus DNA polymerase; Bacillus stearothermophilus DNA polymerase; and archaebacterial DNA polymerases, such as Thermococcus litoralis DNA polymerase (also referred to as Vent_{R}®), Pfu, Pfx, Pwo, and DeepVent_{R}® or a mixture thereof. Other commercially available polymerases DNA polymerases include TaqLA or Expand High Fidelity^{plus} Enzyme Blend (Roche); KlenTaqLA, KlenTaq1, TthLA (Perkin-Elmer), ExTaq® (Takara Shuzo); Elongase® (Life Technologies); TaquenaseT™ (Amersham), TthXL (Perkin Elmer); Advantage™ KlenTaq and Advantage™ Tth (Clontech); TaqPlus® and TaqExtender™ (Stratagene); or mixtures thereof.

In a further embodiment, the present invention includes methods for increasing the specificity of PCR. Preferably, the present invention provides processes and kits for performing a hot-start PCR. The processes and kits utilize the step of generating metal ions, to activate a DNA polymerase enzyme when the temperature of the reaction medium is raised to that enabling metal ion generation by the redox reaction. By performing a hot-start of PCR, the amplification specificity of the target DNA molecules is increased, with minimum or no formation of competitive or inhibitory products.

In a further embodiment, a kit is provided for use in a method of the present invention. Preferably the kit comprises a reaction buffer, a metal compound, an redox agent (e.g. a reducing agent) and a thermostable enzyme, whose activity is dependent on the metal ion in a second oxidation state. Where the thermostable enzyme is a DNA ligase, the kit may further comprise ATP and/or one or more synthetic oligonucleotides. Where the thermostable enzyme is a DNA polymerase, the kit may further comprise dNTPs and/or one or more synthetic oligonucleotides. Preferably the kit comprises a pair of synthetic oligonucleotides or more than one pair or synthetic oligonucleotides for use in a multiplexing PCR reaction. The reaction buffer may also comprise the metal compound.

To aid detection of a PCR product during each cycle of PCR, a technique known in the art as Real-Time PCR can be used. This relies on the detection and quantification of a signal from a fluorescent reporter, the level of which increases in direct proportion to the amount of PCR product being produced.

Therefore, the kit of the present invention may further comprise a fluorescent dye such as SYBR Green®, which binds double stranded DNA. However, since this reporter binds to any double stranded DNA in the reaction e.g. primer-dimers, an overestimation of the product amount may result. Alternatively, the kit may further comprise a reporter probe (e.g. TaqMan®) that contains a fluorescent dye and a quenching dye. These probes hybridize to an internal region of a PCR product and during PCR, when the polymerase enzyme replicates a template on which a reporter probe is bound, the 5′ exonuclease activity of the polymerase cleaves the probe. This separates the fluorescent and quenching dyes resulting in a fluorescent signal. Molecular beacons, which also contain a fluorescent dye and a quenching dye, work on similar principle to TaqMan probes.

In order to prevent premature initiation of the process of the invention, the metal compound can be stored separately to the redox agent. Such storage may be by means of separate vials under conditions appropriate for the storage of reagents for use in PCR or a ligase chain reaction (LCR).

The present reaction composition can be applied to PCR processes as set forth in the Examples.

The principles, methodologies and examples described herein (and below) for controlling metal ion-dependent DNA polymerase activity may be applied in an analogous fashion to control various types of metal ion-dependent enzymes described above.

The following examples illustrate aspects of the invention.

### FIGURES

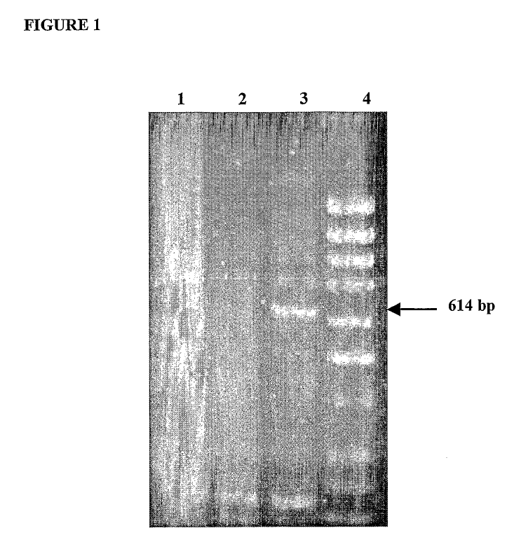

FIG. 1

This figure depicts the electrophoretic analysis of the amplification products obtained when a 614-bp DNA fragment was amplified from 50 ng of Gallus domesticus genomic DNA for 30 cycles. PCR was performed in conventional conditions with ordinary PCR-buffer containing Mg²⁺ (lane 1) or Co²⁺ (lane 2). Lane 3—PCR was performed using controlled generation of Co²⁺. Lane 4—DNA marker. Under these reaction conditions only the controlled generation of divalent ions provided a detectable amount of the desired product (lane 3). Compared to the conventional PCR procedures with Mg²⁺ (lane 1) and Co²⁺ (lane 2), fewer non-specific amplification products were obtained when using controlled generation of Co²⁺ (note the absence of non-specific amplification products in lane 3 compared to lane 1).

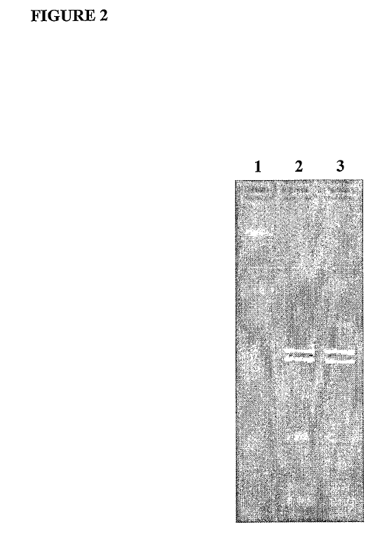

FIG. 2

This figure is an electrophoretic analysis of DNA fragments obtained following restriction endonuclease digestion of pBR322 using TaqI as described in Example 3, indicating that controlled activation of restriction endonuclease activity can be achieved by controlled generation of divalent ions. The enzymatic reaction was performed with (lane 2) and without (lane 1) heat initiation of Co²⁺ generation (note the absence of digestion products in lane 1 compared to lane 2). Lane 3—positive control of endonuclease digestion in presence of Co²⁺.

### EXAMPLES

### Example 1

### The Control of Co²⁺-ions Chemical Generation by Changing Reaction Temperature

Generation of Co²⁺ ions was performed by the reduction-oxidation reaction between hexamminecobalt(III) chloride and ascorbic acid. As a result of the reaction, cobalt(III) was reduced to cobalt(II), and Co²⁺-ions were generated.
2[Co(NH₃)₆]³⁺+C₆H₈O₆→2Co²⁺+2NH₄⁺+10NH₃+C₆H₆O₆

Generation of Co²⁺-ions from [Co(NH₃)₆]³⁺ ions is accompanied by the change of the solution color from yellow to pink. The change of color provides a possibility to monitor the reaction process and the Co²⁺ generation.

The reaction mixture contained: 10 mM hexamminecobalt(III) chloride ([Co (NH₃)₆]Cl₃); 20 mM ascorbic acid (C₆H₈O₆); 100 mM Tris-HCl, pH 9.0 at 25° C. Samples of the reaction mixture (500 μl) were incubated at 25° C., 40° C., 55° C., 70° C., and 85° C. The yellow color of the reaction mixture changed to pink color after the following incubations: 1.5 minutes at 85° C.; 9 minutes at 70° C.; and 80 minutes at 55° C. Incubations at 25° C. and 40° C. for 8 hours did not result in a change of color of the samples. Thus, the reaction of Co²⁺ generation can occur in a controlled manner by heating the reaction mixture.

### Example 2

### Increased Specificity of PCR Using Controlled Generation of Co²⁺ Compared to PCR Performed under Conventional Conditions (in Presence of Divalent Ions)

A) Conventional PCR in Presence of Mg²⁺

A 614-bp DNA fragment was amplified from 50 ng of Gallus domesticus genomic DNA in 30 cycles: 95° C.-30 sec; 58° C.-30 sec; 72° C.-30 sec. The reaction mixture (50 μl) contained: 1.5 mM MgCl₂, 20 mM Tris-HCl (pH 9.0 at 25° C.), 50 mM NH₄Cl, 0.1% Triton X-100, 0.2 mM each dNTP, 25 pmol primer Pr1 (5′-attactcgagatcctggacaccagc), 25 pmol primer Pr2 (5′-attaggatcctgccctctcccca), and 5U Taq DNA polymerase.

B) Conventional PCR in Presence of Co²⁺

A 614 bp DNA fragment was amplified from 50 ng of Gallus domesticus genomic DNA in 30 cycles: 95° C.-30 sec; 58° C.-30 sec; 72° C.-30 sec. The reaction mixture (50 μl) contained: 1 mM CoCl₂, 20 mM Tris-HCl (pH 9.0 at 25° C.), 50 mM NH₄Cl, 0.1% Triton X-100, 0.2 mM each dNTP, 25 pmol primer Pr1 (5′-attactcgagatcctggacaccagc), 25 pmol primer Pr2 (5′-attaggatcctgccctctcccca), and 5U Taq DNA polymerase.

C) PCR Using Controlled Generation of Co²⁺

A 614 bp DNA fragment was amplified from 50 ng of Gallus domesticus genomic DNA in 30 cycles: 95° C.-30 sec; 58° C.-30 sec; 72° C.-30 sec. The reaction mixture (50 μl) contained: 1 mM hexamminecobalt(III) chloride ([Co (NH₃)₆]Cl₃), 2 mM ascorbic acid (C₆H₈O₆), 20 mM Tris-HCl (pH 9.0 at 25° C.), 50 mM NH₄Cl, 0.1% Triton X-100, 0.2 mM each dNTP, 25 pmol primer Pr1 (5′-attactcgagatcctggacaccagc), 25 pmol primer Pr2 (5′-attaggatcctgccctctcccca), and 5U Taq DNA polymerase.

### Example 3

### Control of Restriction Endonuclease Digestion

A) Controlling Restriction Endonuclease Digestion by Co²⁺ Generation

A 100 μl restriction enzyme digestion mixture (100 mM NaCl; 20 mM Tris-HCl (pH 8.5 at 25° C.); 2 μg DNA pBR322; 5 U TaqI restriction endonuclease; 5 mM hexamminecobalt(III) chloride ([Co (NH₃)₆]Cl₃), 7 mM ascorbic acid (C₆H₈O₆)) was prepared. 50 μl samples were removed and placed into two reaction tubes. First tube was incubated at 47° C. for 75 minutes. Second tube was heated to 70° C. for 10 minutes (for heat initiation of Co²⁺ generation), and then it was incubated at 47° C. for 75 minutes.

B) Conventional Restriction Endonuclease Digestion in Presence of Co²⁺ (as a Positive Control of Endonuclease Digestion)

A 100 μl restriction enzyme digestion mixture (100 mM NaCl; 20 mM Tris-HCl (pH 8.5 at 25° C); 2 μg DNA pBR322; 5 U TaqI restriction endonuclease; and 5 mM CoCl₂) was incubated at 47° C. for 75 minutes.

It is to be understood that the above-described methods are merely representative embodiments illustrating the principles of this invention and that other variations in the methods may be devised by those skilled in the art without departing from the spirit and scope of this invention.

1. A process for initiating an enzymatic reaction catalysed by a metal ion-dependent enzyme, comprising the steps of:
a) providing a reaction mixture comprising:
i) a metal compound having a metal atom or metal ion in a first oxidation state;
ii) a redox agent; and
iii) a metal ion-dependent enzyme;
b) heating the mixture of step (a) to react the metal compound with the redox agent in a redox reaction, thereby converting said metal atom or metal ion to a second oxidation state;
wherein, the metal ion-dependent enzyme is activated by the metal atom or metal ion in the second oxidation state.

2. The process according to claim 1, where the metal compound comprises a metal atom or metal ion selected from atoms and ions of: manganese, cadmium, cobalt, copper, iron, molybdenum, nickel, and chromium.

3. The process according to claim 1, wherein the first oxidation state of the metal atom or metal ion is an oxidized state, the redox agent is a reducing agent and the second oxidation state is a reduced state.

4. The process according to claim 1, wherein the first oxidation state of the metal atom or metal ion is a reduced state, the redox agent is an oxidizing agent and the second oxidation state is an oxidized state.

5. The process according to claim 1, wherein the metal atom or metal ion in the second oxidation state is a divalent metal ion.

6. The process according to claim 5, wherein the divalent metal ion is Co²⁺.

7. The process according to claim 1, wherein the redox reaction is selected from:
a reduction of cobalt(III) to cobalt(II),
a reduction of manganese(VII) to manganese(II),
a reduction of manganese(IV) to manganese(II),
a reduction of manganese(III) to manganese(II),
a reduction of chrome(VI) to chrome(II),
a reduction of chrome(III) to chrome(II),
a reduction of iron(III) to iron(II),
a reduction of copper(II) to copper(I),
a reduction of nickel(III) to nickel(II),
a reduction of molybdenum(III) to molybdenum(II),
a reduction of molybdenum(VI) to molybdenum(II),
a reduction of molybdenum(VI) to molybdenum(III),
an oxidation of chromium(II) to chromium(III),
an oxidation of iron(II) to iron(III),
an oxidation of copper(I) to copper(II),
an oxidation of nickel(II) to nickel(III), and
an oxidation of cadmium(I) to cadmium(II).

8. The process according to claim 1, wherein the redox agent is selected from: ascorbic acid, hydroiodic acid, potassium iodide, sodium iodide, ammonium iodide, potassium thiosulfate and sodium thiosulfate.

9. The process according to claim 6, wherein the redox reaction comprises a reaction between a compound of cobalt(III) and ascorbic acid.

10. The process according to claim 6, wherein the redox reaction comprises a reaction between a compound of cobalt(III) and hydroiodic acid.

11. The process according to claim 6, wherein the redox reaction comprises a reaction between hexamminecobalt(III) chloride and one of: ascorbic acid, sodium iodide, potassium iodide or ammonium iodide.

12. The process according to claim 6, wherein the redox reaction comprises a reaction between hexamminecobalt(III) chloride and ascorbic acid.

13. The process according to claim 1, wherein in step (b), the reaction mixture is heated to a temperature greater than 50° C.

14. The process according to claim 1, wherein the metal-ion dependent enzyme is: a polymerase, a ligase, an endonuclease, a kinase, a protease or a combination thereof.

15. The process according to claim 14, wherein the enzyme is a thermostable enzyme.

16. The process according to claim 15, wherein the enzyme is a thermostable DNA ligase.

17. The process according to claim 15, wherein the enzyme is a thermostable DNA polymerase.

18. The process according to claim 17, wherein the enzyme is Taq polymerase or a variant thereof.

19. The process according to claim 1, wherein the enzymatic reaction is, or is part of, a PCR process.

20. A kit for use in the process of claim 1, comprising a reaction buffer, a metal compound having a metal atom or ion in a first oxidation state, a redox agent and a thermostable enzyme.

21. The kit according to claim 20, wherein the first oxidation state of the metal ion is an oxidized state and the redox agent is a reducing agent.

22. The kit according to claim 20 further comprising ATP, and wherein the thermostable enzyme is a DNA ligase.

23. The kit according to claim 20 further comprising dNTPs, and wherein the thermostable enzyme is a DNA polymerase.

24. The kit according to claim 23, further comprising a fluorescent reporter suitable for use in Real-Time PCR.

25. The kit according to claim 20 further comprising one or more synthetic oligonucleotides.

26. The kit according to claim 20, wherein the redox agent and the metal compound are stored separately.

27. The kit according to claim 20, wherein the redox agent and the thermostable enzyme are stored separately.
